(19) United States
(12) Patent Application Publication
(10) Pub. No.: US 2025/0325525 A1
(43) Pub. Date: Oct. 23, 2025

(54) GLP1 TABLET COMPOSITIONS

(71) Applicant: Eli Lilly and Company, Indianapolis, IN (US)

(72) Inventors: Aktham ABURUB, Carmel, IN (US); Matthew Carl ALLGEIER, Fishers, IN (US); Joshua M. HANSON, Indianapolis, IN (US); Siyuan HUANG, Indianapolis, IN (US)

(21) Appl. No.: 18/864,287

(22) PCT Filed: May 10, 2023
(86) PCT No.: PCT/US2023/021641

§ 371 Date: Nov. 8, 2024

Related U.S. Application Data
(60) Provisional application No. 63/340,595, filed on May 11, 2022.

(51) Int. Cl. A61K 31/437; A61K 9/20; A61K 9/28

(52) U.S. Cl. CPC A61K 31/437; A61K 9/2009; A61K 9/2013; A61K 9/2027; A61K 9/2054; A61K 9/28

(57) ABSTRACT

Disclosed herein is a tablet composition comprising 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof, and a pH modifier. In one embodiment, a tablet composition comprises a spray dried dispersion (SDD) of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof, wherein the SDD also comprises a polymer to maintain an amorphous state.

The present invention relates to oral tablet compositions of a GLP-1 receptor agonist, 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one (herein, GLP1RA), or a pharmaceutically acceptable salt thereof. Compositions disclosed herein can be useful for the treatment of type 2 diabetes mellitus (T2D) and in weight management.

Diabetes mellitus is a chronic disorder characterized by hyperglycemia resulting from defects in insulin secretion, insulin action, or both. In T2D, the combined effects of impaired insulin secretion and insulin resistance are associated with elevated blood glucose levels. T2D is an increasingly prevalent disease that frequently leads to declining health and quality of life for patients. Effective oral treatments to manage T2D and/or for use in weight management are desired.

GLP1RA, that is, 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof, is described and claimed in U.S. Pat. No. 10,858,356. The U.S. Pat. No. 10,858,356 patent generally describes oral compositions.

GLP1RA may be prepared as a pharmaceutically acceptable salt. One salt of GLP1RA is a hemi-calcium hydrate, 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate, (herein “GLP1RA-Ca”) with the structure as shown below.

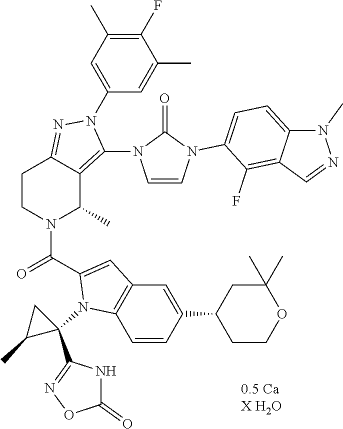

GLP1RA is a poorly permeable and poorly soluble weak acid with a pKa of 5.1. GLP1RA has very low aqueous solubility across the physiologic pH range as well as in simulated physiological fluids. GLP1RA and its pharmaceutically acceptable salts are observed to have a strong pH dependent solubility profile contributing to challenges such as variability in absorption and consequently in pharmacokinetic performance and potential food effects. There is a desire for GLP1RA, including but not limited to, GLP1RA-Ca, tablet compositions providing reliable PK performance in a patient friendly dosage form, with minimal potential for drug-drug interactions and reduced or no food effects. A GLP1RA composition to enhance solubility and dissolution rate of the active substance in a tablet dosage form may be desired. A pharmaceutically elegant dosage form to deliver an effective amount of active GLP1RA to the targeted portion of the gastrointestinal tract, while small enough to facilitate patient swallowing is desired.

Compositions described herein provide desired properties. In an aspect, the use of a sprayed dried dispersion (SDD) of the GLP1RA, or a pharmaceutically acceptable salt thereof, together with a pH modifier, as described herein, contributes to the desired properties. In an aspect, the specific particle sizes of the SDD and the particular compositions as described provide the desired properties. In an aspect, compositions disclosed herein provide desirable pharmacokinetic performances and deliver an effective amount of active GLP1RA to the targeted portion of the gastrointestinal tract. In an aspect, disclosed herein is an elegant dosage form that is convenient for patients to take without the limitation of food or water restrictions.

Solid oral formulations provided herein can be useful for patients in need of treatment for T2D. Solid oral formulations provided herein can be useful for patients in need of chronic weight management.

In an embodiment is a tablet composition comprising:
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier.

In an embodiment, the tablet composition disclosed herein is an oral solid composition.

In an embodiment is a composition wherein a pH modifier is selected from the group consisting of calcium carbonate, magnesium carbonate, sodium bicarbonate, sodium carbonate, magnesium hydroxide, calcium hydroxide, magnesium oxide, and a mixture thereof.

In an embodiment is a solid oral tablet composition comprising
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier; wherein the pH modifier is selected from the group consisting of calcium carbonate, anhydrous calcium carbonate, sodium bicarbonate, anhydrous sodium bicarbonate, sodium carbonate, anhydrous sodium carbonate, sodium carbonate hydrate, magnesium hydroxide, and anhydrous magnesium hydroxide.

In an embodiment, a pH modifier is sodium carbonate. In an embodiment, a pH modifier is anhydrous. In an embodiment, a pH modifier is anhydrous sodium carbonate.

In an embodiment, a pH modifier is sodium carbonate hydrate.

In an embodiment, a pH modifier is sodium bicarbonate. In an embodiment, a pH modifier is anhydrous. In an embodiment, a pH modifier is anhydrous sodium bicarbonate.

In an embodiment is a tablet composition comprising 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier; wherein the pH modifier is anhydrous sodium carbonate.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier; and
a super disintegrant.

In an embodiment, a super disintegrant is selected from the group consisting of croscarmellose sodium, crospovidone, sodium starch glycolate, and a mixture thereof. In an embodiment, a super disintegrant is selected from the group consisting of croscarmellose sodium and crospovidone.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier; and
a lubricant.

In an embodiment, a lubricant is selected from the group consisting of calcium stearate, magnesium stearate, polyethylene glycol, sodium stearyl fumarate, stearic acid, talc, and a mixture thereof. In an embodiment, a lubricant comprises magnesium stearate. In an embodiment, a lubricant is magnesium stearate.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier;
a super disintegrant; and
a lubricant.

In an embodiment is a tablet composition wherein the composition comprises 3-[(1S,2S)-1-[5-[(4S)-2,2-dimethyloxan-4-yl]-2-[(4S)-2-(4-fluoro-3,5-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier;
a super disintegrant selected from the group consisting of croscarmellose sodium and crospovidone; and
a lubricant which is magnesium stearate.

In an embodiment is a tablet composition wherein the composition comprises 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier; and
crospovidone.

In an embodiment, a tablet composition comprises an enteric coating. In an embodiment, a tablet composition comprises an immediate release coating. In an embodiment, a tablet composition comprises both an immediate release coating and an enteric coating.

In an embodiment, a tablet composition comprises 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; and a pH modifier.

In an embodiment, a tablet composition comprises 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one; in an amount of about 0.5 to about 75 mg per tablet composition.

In an embodiment, a tablet composition comprises 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one; in an amount of about 0.5 to about 60 mg; or more specifically, about 0.5 to about 45 mg; or more specifically, about 0.5 to about 36 mg; or even more specifically, about 0.8 to about 36 mg; per tablet composition.

In an embodiment, a tablet composition comprises 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 0.5 to about 82 mg; or more specifically, about 0.5 to about 75 mg; or more specifically, about 0.5 to about 60 mg; or more specifically, about 0.5 to about 45 mg; or more specifically, about 0.8 to about 45 mg; or even more specifically, about 1 to about 36 mg; (all amounts are on free acid basis); per tablet composition.

As described herein, the conversion factor for GLP1RA/GLP1RA-Ca (i.e., the hemicalcium salt hydrate of GLP1RA) is assumed to be around 0.91. As a skilled artisan readily appreciates, the exact conversion rate may change slightly depending on the actual content of the hydrate.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 0.5 mg to about 75 mg (on a free acid basis);
a pH modifier selected from the group consisting of calcium carbonate, sodium bicarbonate, sodium carbonate, sodium carbonate hydrate, magnesium hydroxide, and a mixture thereof; in an amount of about 5 mg to about 15 mg;
a super disintegrant selected from the group consisting of croscarmellose sodium and crospovidone; in an amount of about 2 mg to about 25 mg; and
a lubricant selected from the group consisting of calcium stearate, magnesium stearate, polyethylene glycol, sodium stearyl fumarate, stearic acid, talc, and a mixture thereof; in an amount of about 0.5 mg to about 5 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 0.7 mg to about 60 mg (on a free acid basis);
a pH modifier selected from the group consisting of calcium carbonate, sodium bicarbonate, sodium carbonate, sodium carbonate hydrate, magnesium hydroxide, and a mixture thereof; in an amount of about 5 mg to about 15 mg;
a super disintegrant selected from the group consisting of croscarmellose sodium and crospovidone; in an amount of about 2 mg to about 25 mg; and
a lubricant which is magnesium stearate; in an amount of about 0.5 mg to about 5 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 0.8 mg to about 45 mg (on a free acid basis);
a pH modifier selected from the group consisting of calcium carbonate, sodium bicarbonate, sodium carbonate, sodium carbonate hydrate, magnesium hydroxide, and a mixture thereof; in an amount of about 5 mg to about 15 mg;
a super disintegrant selected from the group consisting of croscarmellose sodium and crospovidone; in an amount of about 2 mg to about 17.5 mg; and
a lubricant which is magnesium stearate; in an amount of about 1 mg to about 2.5 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 1 to about 36 mg (on a free acid basis);
a pH modifier selected from the group consisting of calcium carbonate, sodium bicarbonate, sodium carbonate, sodium carbonate hydrate, and magnesium hydroxide, and a mixture thereof; in an amount of about 5 mg to about 10 mg;
a super disintegrant selected from the group consisting of croscarmellose sodium and crospovidone; in an amount of about 5 mg to about 20 mg; and
a lubricant which is magnesium stearate; in an amount of about 1 mg to about 2.5 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 1 mg to about 30 mg (on a free acid basis);
a pH modifier selected from the group consisting of anhydrous calcium carbonate, anhydrous sodium bicarbonate, anhydrous sodium carbonate, sodium carbonate hydrate, and anhydrous magnesium hydroxide; in an amount of about 6 mg to about 8 mg;
a super disintegrant selected from the group consisting of croscarmellose sodium and crospovidone; in the amount of about 10 mg to about 18 mg; and
a lubricant which is magnesium stearate; in an amount of about 1.5 mg to about 2 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 0.8 mg to about 45 mg (on a free acid basis);
a pH modifier which is anhydrous sodium carbonate; in an amount of about 8 mg;
a super disintegrant which is crospovidone; in an amount of about 17.5 mg; and
a lubricant which is magnesium stearate; in an amount of about 2 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 1 mg to about 36 mg (on a free acid basis);
a pH modifier which is anhydrous sodium carbonate; in an amount of about 8 mg;
a super disintegrant which is crospovidone; in an amount of about 17.5 mg; and
a lubricant which is magnesium stearate; in an amount of about 2 mg.

In an embodiment is a tablet composition wherein the composition comprises
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 45 mg to about 60 mg (on a free acid basis);
a pH modifier which is anhydrous sodium carbonate; in an amount of about 8 mg;
a super disintegrant which is crospovidone; in an amount of about 17.5 mg; and
a lubricant which is magnesium stearate; in an amount of about 2 mg.

In an embodiment, GLP1RA or a pharmaceutically acceptable salt thereof is prepared into a spray dried dispersion (SDD) for use as the active drug in a tablet composition. In an embodiment, an SDD of GLP1RA or a pharmaceutically acceptable salt thereof is prepared under the conditions as described in Preparation 2 and Preparation 3 below.

In an embodiment, the GLP1RA (“3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one”) or GLP1RA-Ca (“3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate”) is present as an SDD preparation in a composition described herein.

In general, an SDD preparation disclosed herein comprises GLP1RA, or a pharmaceutically acceptable salt thereof, and a polymer as a balance component to maintain an amorphous state of the GLP1RA, or a pharmaceutically acceptable salt thereof. In an embodiment, the polymer is selected from the group consisting of polyvinyl pyrrolidone (also known as “povidone” or “PVP”) and polyvinyl pyrrolidone vinyl acetate (also known as “copovidone” or “PVP-VA”). In an embodiment, the polymer is PVP-VA. In an embodiment, the polymer is PVP.

In an embodiment where the weight percentage of the GLP1RA, or a pharmaceutically acceptable salt thereof, in an SDD preparation is specified, the balance component of the SDD is a polymer selected from PVP and PVP-VA, that is, the total weight percentage of the GLP1RA, or a pharmaceutically acceptable salt thereof, and the polymer is 100 wt %. In an embodiment, the polymer is PVP-VA. In an embodiment, the polymer is PVP.

In an embodiment, the SDD preparation comprises about 20 wt % to about 40 wt % of GLP1RA or GLP1RA-Ca and the balance is composed of PVP-VA. In an embodiment, the SDD preparation comprises about 30 wt % to about 35 wt % of GLP1RA or GLP1RA-Ca and the balance is composed of PVP-VA. In an embodiment, the SDD preparation comprises about 30 wt % of GLP1RA or GLP1RA-Ca and the balance is composed of PVP-VA. In an embodiment, the SDD preparation comprises about 30 wt % of GLP1RA-Ca and the balance is composed of PVP-VA.

In an embodiment, the mean particle size of the GLP1RA or GLP1RA-Ca SDD is about 5 μm to about 150 μm in diameter. In an embodiment, the mean particle size of the SDD is about 5 μm to about 113 μm in diameter. In an embodiment, the mean particle size of the SDD is about 40 μm to about 65 μm in diameter. In an embodiment, the mean particle size of the SDD is about 40 μm to about 50 μm in diameter. In an embodiment, the mean particle size of the SDD is about 5 μm to about 25 μm in diameter.

In an embodiment is a tablet composition comprising:
an SDD comprising 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier.

In an embodiment, the pH modifier is selected from the group consisting of calcium carbonate, magnesium carbonate, sodium bicarbonate, sodium carbonate, magnesium hydroxide, calcium hydroxide, magnesium oxide, and a mixture thereof. In an embodiment, the pH modifier is sodium bicarbonate. In an embodiment, the pH modifier is sodium carbonate. In an embodiment, the pH modifier is anhydrous sodium carbonate.

In an embodiment is a tablet composition comprising:
an SDD of about 20 wt % to about 40 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier selected from the group consisting of calcium carbonate, magnesium carbonate, sodium bicarbonate, sodium carbonate, magnesium hydroxide, calcium hydroxide, magnesium oxide, and a mixture thereof.

In an embodiment is a tablet composition comprising:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier selected from the group consisting of sodium bicarbonate and sodium carbonate.

In an embodiment is a tablet composition comprising:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; wherein the SDD has a mean particle size of about 5 μm to about 113 μm in diameter; and
a pH modifier selected from the group consisting of sodium bicarbonate and sodium carbonate.

In an embodiment is a tablet composition comprising:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; and the balance is composed of a polymer selected from PVP-VA and PVP; wherein the SDD has a mean particle size of about 5 μm to about 113 μm in diameter; and
a pH modifier selected from the group consisting of sodium bicarbonate and sodium carbonate.

In an embodiment is a tablet composition comprising:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 0.5 mg to about 60 mg (on a free acid basis); and the balance is composed of a polymer selected from PVP-VA and PVP; and
a pH modifier selected from the group consisting of sodium bicarbonate and sodium carbonate.

In an embodiment is a tablet composition comprising:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 0.8 mg to about 45 mg (on a free acid basis); and the balance is composed of a polymer which is PVP-VA; and
a pH modifier selected from the group consisting of sodium bicarbonate and sodium carbonate.

In an embodiment is a tablet composition comprising:
an SDD of about 30 wt % to about 35 wt % 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of about 0.5 mg to about 60 mg (on a free acid basis); and the balance is composed of a polymer which is PVP-VA; and
a pH modifier selected from the group consisting of sodium bicarbonate and sodium carbonate; in an amount of about 1 mg to about 100 mg.

In an embodiment of the tablet compositions described above, the SDD has a mean particle size of about 5 μm to about 113 μm in diameter; and
the pH modifier is sodium carbonate.

In an embodiment of the tablet compositions described above, the composition further comprises:
a super disintegrant selected from the group consisting of croscarmellose sodium and crospovidone; and
a lubricant.

In an embodiment of the tablet compositions described above, the super disintegrant is crospovidone in an amount of about 2 mg to about 250 mg; and
the lubricant is magnesium stearate in an amount of about 0.1 mg to about 10 mg.

In an embodiment, disclosed herein is a tablet composition selected from the group consisting of composition 1 (Comp. 1), composition 2 (Comp. 2), composition 3 (Comp. 3), composition 4 (Comp. 4), composition 5 (Comp. 5), composition 6 (Comp. 6), composition 7 (Comp. 7), and composition 8 (Comp. 8), as shown in the following table, wherein the amount of each ingredient of a particular composition is shown as the target amount for that composition. In one embodiment, GLP1RA-Ca is used as the active ingredient and the amount of each GLP1RA in the compositions is on the free acid basis.

| | Target Amount and Variable Ranges in Parenthesis (mg/tablet) | | | |
|---|---|---|---|---|
| | Comp. 1 | Comp. 2 | Comp. 3 | Comp. 4 |
| | 60 mg GLP1RA | 45 mg GLP1RA | 30 mg GLP1RA | 20 mg GLP1RA |
| | (A: 54-66 mg) | (A: 40-50 mg) | (A: 27-33 mg) | (A: 18-22 mg) |
| Ingredient | (B: 57-63 mg) | (B: 42-47 mg) | (B: 28-32 mg) | (B: 19-21 mg) |
| SDD (30 wt % | 200 | 150 | 100 | 67 |
| of GLP1RA) | (A: 180-220) | (A: 135-165) | (A: 90-110) | (A: 60-73) |
| | (B: 190-210) | (B: 143-158) | (B: 95-105) | (B: 63-70) |
| Filler | 332 | 382 | 282 | 188 |
| | (A: 265-400) | (A: 305-458) | (A: 226-338) | (A: 150-225) |
| | (B: 299-365) | (B: 344-420) | (B: 254-310) | (B: 169-207) |
| Disintegrant | 121 | 121 | 87 | 58 |
| | (A: 97-145) | (A: 97-145) | (A: 70-105) | (A: 47-70) |
| | (B: 109-134) | (B: 109-134) | (B: 109-134) | (B: 52-64) |
| pH Modifier | 57 | 57 | 41 | 27 |
| | (A: 46-69) | (A: 46-69) | (A: 33-49) | (A: 22-33) |
| | (B: 51-63) | (B: 51-63) | (B: 37-45) | (B: 25-30) |
| Lubricant | 3.6 | 3.6 | 2.6 | 1.7 |
| | (A: 2.9-4.3) | (A: 2.9-4.3) | (A: 2.1-3.1) | (A: 1.4-2.1) |
| | (B: 3.2-3.9) | (B: 3.2-3.9) | (B: 2.3-2.8) | (B: 1.6-1.9) |
| Glidant | 0-20 | 0-20 | 0-15 | 0-10 |
| (optional) | | | | |
| Total tab. wt. | 714 mg | 714 mg | 513 mg | 342 mg |

| | Target Amount and Variable Ranges in Parenthesis (mg/tablet) | | | |
|---|---|---|---|---|
| | Comp. 5 | Comp. 6 | Comp. 7 | Comp. 8 |
| | 10 mg | 5 mg | 2.5 mg | 0.8 mg |
| | (A: 9-11 mg) | (A: 4.5-5.5 mg) | (A: 2.2-2.8 mg) | (A: 0.6-1 mg) |
| Ingredient | (B: 9.5-10.5 mg) | (B: 4.6-5.3 mg) | (B: 2.3-2.6 mg) | (B: 0.7-0.9 mg) |
| SDD (30 wt % | 33 | 16.7 | 8.3 | 2.8 |
| of GLP1RA) | (A: 30-36) | (A: 15-18) | (A: 7.5-9.1) | (A: 2.1-3.3) |
| | (B: 31-34) | (B: 16-17) | (B: 7.8-8.6) | (B: 2.3-3.0) |
| Filler | 94 | 47 | 61 | 67 |
| | (A: 75-113) | (A: 38-56) | (A: 49-7.3) | (A: 53-80) |
| | (B: 85-103) | (B: 42-52) | (B: 55-67) | (B: 60-73) |
| Disintegrant | 29 | 14.5 | 8.5 | 8.5 |
| | (A: 23-35) | (A: 12-17) | (A: 6.8-10) | (A: 6.8-10) |
| | (B: 26-32) | (B: 13-16) | (B: 7.7-9.4) | (B: 7.7-9.4) |
| pH Modifier | 14 | 6.8 | 6.8 | 6.8 |
| | (A: 11-16) | (A: 5.5-8.2) | (A: 5.4-8.2) | (A: 5.4-8.2) |
| | (B: 12-15) | (B: 6.2-7.5) | (B: 6.2-7.5) | (B: 6.2-7.5) |
| Lubricant | 0.8 | 0.4 | 0.4 | 0.4 |
| | (A: 0.7-1.0) | (A: 0.3-0.5) | (A: 0.3-0.5) | (A: 0.3-0.5) |
| | (B: 0.7-0.9) | (B: 0.35-0.4) | (B: 0.35-0.4) | (B: 0.35-0.4) |
| Glidant | 0-10 | 0-5 | 0-5 | 0-5 |
| (optional) | | | | |
| Total tab. wt. | 171 mg | 85 mg | 85 mg | 85 mg |

As one skilled in pharmaceutical formulation can appreciate, the amount of one or more ingredients of a composition can be changed within certain limits while the desired properties of a composition can be maintained. Additionally, when the amount of one ingredient is changed, the amount of one or more other ingredients can be adjusted accordingly within certain ranges to keep the total weight of a composition constant and maintain the desired properties of the composition.

In an embodiment of a particular composition shown in the above table, the amount of each ingredient is within the respective variable A range as indicated in parenthesis from the target amount while the desired properties of the corresponding composition are maintained.

In an embodiment of a particular composition shown in the above table, the amount of each ingredient is within the respective variable B range as indicated in parenthesis from the target amount while the desired properties of the corresponding composition are maintained.

In an embodiment is a process for preparing a composition as claimed herein comprising a spray dried dispersion process.

In an embodiment is a tablet composition wherein the composition comprises:
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of between about 0.5 mg to about 75 mg, on a free acid basis;
a pH modifier selected from the group consisting of calcium carbonate, sodium bicarbonate, sodium carbonate, sodium carbonate hydrate, magnesium hydroxide, and a mixture thereof; in an amount of about 1 mg to about 150 mg;
a super disintegrant selected from the group consisting of croscarmellose sodium and crospovidone; in an amount of about 1 mg to about 250 mg; and
a lubricant which is magnesium stearate; in an amount of about 0.1 mg to about 10 mg.

In an embodiment is a tablet composition as claimed by claim 21 wherein the composition comprises:
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of between about 0.7 mg to about 60 mg, on a free acid basis;
a pH modifier selected from the group consisting of calcium carbonate, sodium bicarbonate, sodium carbonate, sodium carbonate hydrate, magnesium hydroxide, and a mixture thereof; in an amount of about 2 mg to about 100 mg;
a super disintegrant selected from the group consisting of croscarmellose sodium and crospovidone; in an amount of about 2 mg to about 200 mg; and
a lubricant which is magnesium stearate; in an amount of about 0.1 mg to about 2.5 mg.

In an embodiment is a tablet composition wherein the composition comprises:
an SDD of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier.

In an embodiment is a tablet composition wherein the composition comprises:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 0.5 mg to about 75 mg, on a free acid basis; and the balance of the SDD is composed of PVP-VA; and
a pH modifier selected from the group consisting of sodium bicarbonate and sodium carbonate; in an amount of between about 1 mg to about 150 mg.

In an embodiment is a tablet composition wherein the composition comprises:
an SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or the 0.5 Ca hydrate thereof; in an amount of between about 0.7 mg to about 60 mg, on a free acid basis; and the balance of the SDD is composed of PVP-VA; and
a pH modifier selected from the group consisting of sodium bicarbonate and sodium carbonate; in an amount of between about 5 mg to about 100 mg.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or the 0.5 Ca hydrate thereof; in an amount of between about 1.7 mg to about 250 mg of the SDD; and the balance of the SDD is composed of PVP-VA; wherein the SDD is between about 2 wt % to about 25 wt % of the total tablet weight; and
a pH modifier which is sodium carbonate; in an amount of between about 6 mg to about 100 mg; wherein the pH modifier is between about 5 wt % to about 15 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of between about 1.7 mg to about 14.3 mg of the SDD; and the balance of the SDD is composed of PVP-VA; wherein the SDD is between about 2 wt % to about 16.9 wt % of the total tablet weight; and
a pH modifier which is sodium carbonate; in an amount of about 6.8 mg; wherein the pH modifier is about 8 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of between about 16.7 mg to about 120 mg of the SDD; and the balance of the SDD is composed of PVP-VA; wherein the SDD is about 19.5 wt % of the total tablet weight; and
the pH modifier which is sodium carbonate; in an amount of between about 6.8 mg to about 49.2 mg; wherein the pH modifier is about 8 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; in an amount of between about 150 mg to about 250 mg of the SDD; and the balance of the SDD is composed of PVP-VA; wherein the SDD is between about 21 wt % to about 25 wt % of the total tablet weight; and
a pH modifier which is sodium carbonate; in an amount of between about 57.1 mg to about 80 mg; wherein the pH modifier is about 8 wt % of the total tablet weight.

In an embodiment of the tablet compositions as described above, the SDD has a mean particle size of about 5 μm to about 113 μm in diameter.

In an embodiment is a tablet composition, wherein the composition further comprises: a super disintegrant selected from the group consisting of croscarmellose sodium and crospovidone.

In an embodiment is a tablet composition wherein:
the super disintegrant is crospovidone in an amount of between about 8.5 mg to about 170 mg; wherein the super disintegrant is between about 10 wt % to about 17 wt % of the total tablet weight.

In an embodiment is a tablet composition wherein the composition further comprises:
a filler which is MCC; in an amount of between about 67 mg to about 495 mg; wherein the filler is between about 49.5 wt % to about 79.5 wt % of the total tablet weight.

In an embodiment is a tablet composition, wherein the composition further comprises:
a lubricant which is magnesium stearate; in an amount of between about 0.4 mg to about 5 mg; wherein the lubricant is between about 0.1 wt % to about 1 wt % of the total tablet weight.

In an embodiment is a tablet composition, wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 19.5 wt % of the total tablet weight;
a pH modifier which is sodium carbonate; wherein the pH modifier is about 8 wt % of the total tablet weight;
a super disintegrant which is crospovidone; wherein the super disintegrant is about 17 wt % of the total tablet weight; and
a filler which is MCC; wherein the filler is about 55 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 25 wt % of the total tablet weight;
a pH modifier which is sodium carbonate; wherein the pH modifier is about 8 wt % of the total tablet weight;
a super disintegrant which is crospovidone; wherein the super disintegrant is about 17 wt % of the total tablet weight; and
a filler which is MCC; wherein the filler is about 49.5 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 21 wt % of the total tablet weight;
a pH modifier which is sodium carbonate; wherein the pH modifier is about 8 wt % of the total tablet weight;
a super disintegrant which is crospovidone; wherein the super disintegrant is about 17 wt % of the total tablet weight; and
a filler which is MCC; wherein the filler is about 53.5 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 16.9 wt % of the total tablet weight;
a pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 64.6 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 11.8 wt % of the total tablet weight;
a pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 69.7 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; and the balance of the SDD is composed of PVP-VA; wherein the SDD is about 9.8 wt % of the total tablet weight;
a pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 71.7 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 3.9 wt % of the total tablet weight;
a pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 77.6 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 3.2 wt % of the total tablet weight;
a pH modifier which is sodium carbonate; wherein the pH modifier is about 8 wt % of the total tablet weight;
a super disintegrant which is crospovidone; wherein the super disintegrant is about wt % of the total tablet weight; and
a filler which is MCC; wherein the filler is about 78.3 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 2.7 wt % of the total tablet weight;
a pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 78.8 wt % of the total tablet weight.

In an embodiment is a tablet composition wherein the composition comprises:
an SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]2-methylcyclopropyl]-4H-1,2,4-oxadiazol-5-one, 0.5 Ca hydrate; and the balance of the SDD is composed of PVP-VA; wherein the SDD is about 2.0 wt % of the total tablet weight;
a pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 79.5 wt % of the total tablet weight.

In an embodiment is a tablet composition as described above wherein the composition further comprises a lubricant which is magnesium stearate; wherein the lubricant is about 0.5 wt % of the total tablet weight.

In an embodiment is a composition as described above wherein the composition further comprises an immediate release coating.

In an embodiment is a tablet composition as described above wherein the composition further comprises an enteric coating over the immediate release coating.

Pharmaceutical compositions using amorphous forms of an active, have been prepared using spray dried dispersions (SDD), to develop dosage forms for poorly soluble drugs. However, this SDD approach is complicated by the presence of polymeric functional excipients that may be required to maintain the drug in an amorphous state. Compositions providing acceptable pharmacokinetic performance and desired food effects profile can be especially challenging for molecules possessing strong pH dependent solubility profiles. GLP1RA and GLP1RA-Ca exhibit such pH dependent solubility profile. Weak acids can present challenges in ensuring dissolution of the active substance is achieved in the desired portion of the gastrointestinal tract, particularly the stomach, where low pH could retard dissolution of weakly acidic drugs. Certain excipients may become unstable when added to a tablet composition described herein. In an embodiment, an excipient is stable in a tablet composition with pH modifier.

As used herein, a pH modifier means an inorganic basic salt including, but not limited to, calcium carbonate, magnesium carbonate, sodium bicarbonate, sodium carbonate, magnesium hydroxide, calcium hydroxide, magnesium oxide including both anhydrous and hydrated forms of the respective salts capable of raising the micro pH during tablet disintegration and dissolution.

In an embodiment, a GLP1RA or a pharmaceutically acceptable salt composition comprises a pH modifier that is sodium carbonate. In an embodiment, a GLP1RA or a pharmaceutically acceptable salt composition comprises a pH modifier that is sodium carbonate in its anhydrous form.

In an embodiment is a composition comprising GLP1RA, or a pharmaceutically acceptable salt thereof, and a pH modifier that is selected from the group consisting of calcium carbonate, sodium bicarbonate, sodium carbonate, magnesium hydroxide, and a mixture thereof.

In an embodiment is a composition comprising GLP1RA, or a pharmaceutically acceptable salt thereof, and a pH modifier selected from the group consisting of calcium carbonate, anhydrous calcium carbonate, sodium bicarbonate, anhydrous sodium bicarbonate, sodium carbonate, anhydrous sodium carbonate, magnesium hydroxide, and anhydrous magnesium hydroxide.

In an embodiment, a tablet composition comprising GLP1RA or a pharmaceutically acceptable salt thereof, and a pH modifier is uncoated.

In an embodiment, a tablet composition is prepared as an immediate release (IR) dosage form that releases GLP1RA or a pharmaceutically acceptable salt thereof in the gastric compartment. Excessive amounts of pH modifier in the tablet may affect absorption of a co-administered medicine whose absorption is pH sensitive. In an embodiment, a pH modifier is in sufficiently low amounts such that it will not raise the bulk gastric pH during dissolution but sufficient to facilitate the dissolution of the GLP1RA or a pharmaceutically acceptable salt thereof. An immediate release dosage form with sufficiently low amount of a pH modifier may be desired to minimize potential for elevated stomach pH and drug-drug interaction (DDI) with a co-administered drug.
In an embodiment, a tablet composition comprising GLP1RA or a pharmaceutically acceptable salt thereof, and a pH modifier is coated with an immediate release coating.

As used herein, the term “immediate release coating” means a coating that is intended primarily for aesthetics and is not intended to alter disintegration and/or dissolution of the tablet. As used herein, appropriate immediate release coatings may be selected from known coatings, for example, but not limited to, Opadry® Clear (03K19229) (Colorcon Inc). As is common for moisture sensitive SDD based formulations, protection from moisture may be desired, to ensure product functionality and elegance throughout shelf life. This protection may be achieved through packaging, as well as utilization of coating materials to minimize moisture uptake of the tablet. One known immediate release coating with this functionality is Opadry® ambII (Colorcon).

In an embodiment is a composition comprising GLP1RA, or a pharmaceutically acceptable salt thereof; a pH modifier; and an immediate release coating.

In an embodiment, a tablet composition comprising GLP1RA, or a pharmaceutical salt thereof, and a pH modifier is prepared as a delayed release (DR) dosage form further comprising an enteric coating. A composition further comprising an enteric coating may release in the intestine at a pH of 5.5 or greater. Release in the intestine may be desired for GLP1RA, or a pharmaceutical salt thereof, to limit risks potentially associated with the presence of a pH modifier in the tablet. A DR composition may minimize potential food effect, and may have other favorable effects to the pharmacokinetic profile, for example, but not limited to, overall exposure and modulation of Cmax.

As used herein, “enteric coating” means a functional coating that is intended to prevent tablet disintegration and/or dissolution in the gastric environment but dissolve in the intestinal environment.

In an embodiment is a composition comprising GLP1RA, or a pharmaceutically acceptable salt thereof; a pH modifier; and an enteric coating.

In an embodiment is a tablet composition comprising GLP1RA, or a pharmaceutical salt thereof; a pH modifier; and a super disintegrant.

In an embodiment is a tablet composition comprising GLP1RA, or a pharmaceutical salt thereof; a pH modifier; a super disintegrant; and a lubricant.
In an embodiment is a tablet composition comprising GLP1RA, or a pharmaceutically acceptable salt thereof; a pH modifier; a super disintegrant; and an enteric coating on the tablet.

In an embodiment is a tablet composition comprising GLP1RA, or a pharmaceutically acceptable salt thereof; a pH modifier; a super disintegrant; and an immediate release coating on the tablet.

As used herein, terms “super disintegrant” and “disintegrant” are interchangeable and mean an excipient to facilitate desirable dissolution of the GLPRA, or pharmaceutical salt thereof, including but not limited to, croscarmellose sodium and crospovidone. The term “super disintegrant” refers to an excipient that preferably facilitates dissolution of the GLP1RA, or pharmaceutical salt thereof, within 30 to about 60 minutes. In an embodiment, a tablet composition comprising GLP1RA, or a pharmaceutical salt thereof; a pH modifier; and a super disintegrant selected from the group consisting of croscarmellose sodium and crospovidone.

In an embodiment is a tablet composition comprising GLP1RA, or a pharmaceutical salt thereof; a pH modifier, and crospovidone.

In an embodiment, a desired tablet composition provides reliable PK performance, in a patient friendly dosage form. Patient friendly tablet composition dosage forms are desired with acceptable size to facilitate swallowing during daily administration, have minimal potential for drug-drug interactions (DDI) with other concomitant medication, and have minimal use restrictions, including minimizing food effects, avoiding restrictions on time of administration, and avoiding restriction on the volume of liquid used to aide in swallowing.

Tablet compositions may release either in the gastric compartment or intestine (enteric) through an erosional mechanism. A dosage form of this invention provides the potential to improve absorption/PK performance. Dosage forms provided herein can reduce undesired drug-drug interactions.

Certain abbreviations are defined as follows: “cfm” refers to cubic feet/minute; “Cmax” refers to the maximum plasma concentration that a drug achieves in tested area after the drug has been administered and prior to the administration of a second dose; “DDI” refers to drug-drug interactions; “DR” refers to delayed release; “EtOH” refers to ethanol or ethyl alcohol; “FaSSiF” refers to fasted state simulated intestinal fluid; “FaSSGF” refers to fasted state simulated gastric fluid; “FeSSIF” refers to fed state simulated intestinal fluid; “hr” refers to hour; “hrs” refers to hours; “IR” refers to immediate release; “PK” refers to pharmacokinetics; “MeOH” refers to methanol or methyl alcohol; “rpm” refers to revolutions per minute; “PVP-VA” refers to polyvinylpyrolido/vinyl acetate copolymer; “SDD” refers to spray dried dispersion “SIF” refers to simulated intestinal fluid; “T2D” refers to type 2 diabetes; “THF” refers to tetrahydrofuran; “USP” refers to United States Pharmacopeia; “wt %” refers to mass of desired material/total mass; and XRPD refers to X-ray powder diffraction. As used herein “prep” means to preparation.

A skilled artisan may prepare 3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca, spray dried dispersion (SDD) preparation under a variety of processing conditions and with varying equipment to produce material of suitable quality and critical characteristics.

In an embodiment, a GLP1RA or GLP1RA-Ca in an SDD preparation is amorphous as measured via XRPD, of the desired wt % of drug (30%) and PVP-VA (70%), and of a particle size that can be isolated and forward processed and is acceptably free of process related impurities and excessive residual solvents.

In an embodiment, amorphous solid dispersion may be used in the preparation process. In an embodiment, the particle size is from about 5 to about 113 μm for use in the preparing the tablet composition.

Capsules are prepared for assays herein, utilizing conventional encapsulation methods.

### PREPARATION 1

### 3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate

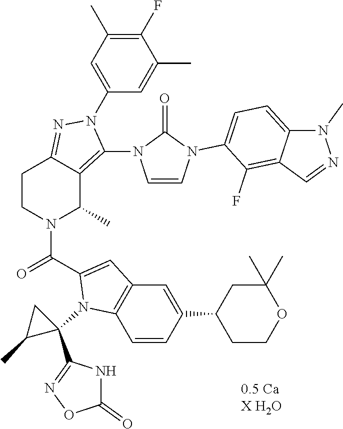

Example 1 may be prepared as described in WO18/056453.

The title compound has alternative names. For example, it is also known as the hemicalcium salt hydrate of orforglipron.

Another name for the title compound is 1,2,4-oxadiazol-5 (2H)-one, 3-[(1S,2S)-1-[2-[[(4S)-2-(4-fluoro-3,5-dimethylphenyl)-3-[3-(4-fluoro-1-methyl-1H-indazol-5-yl)-2,3-dihydro-2-oxo-1H-imidazol-1-yl]-2,4,6,7-tetrahydro-4-methyl-5H-pyrazolo[4,3-c] pyridin-5-yl]carbonyl]-5-[(4S)-tetrahydro-2,2-dimethyl-2H-pyran-4-yl]-1H-indol-1-yl]-2-methylcyclopropyl]-, calcium salt (2:1), hydrate.

### PREPARATION 2

### 3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate, SDD preparation

30% 3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca SDD is prepared by dissolving 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate (8.7 g, 92% potency) and PVP-VA, (also known as copovidone, CAS #25086-89-9;) (18.7 g) in EtOH (200 mL) at room temperature. Following dissolution of the solids the solution is spray dried on a conventional spray dryer with a pressure nozzle. The following parameters in Table 1 are used to create the dispersion using a laboratory scale spray drier. Spray drying can begin when the spray dryer temperature is above 33° C. Collect the material and dry under vacuum at 50° C. overnight to give the title compound (21.99 g, 7.0 g, 92% potency, 80% recovery) and observe via photomicroscopy that the material is microscopically non birefringent particles that are approximately 5-25 μm in diameter.

### SDD Parameters

TABLE 1

| Process Parameter | Value | |
|---|---|---|
| Aspirator | 100% | |
| Nebulization Flow | 37 mm | (~400 L/hr) |
| Solution flow rate | 40% | (12.5 mL/min) |
| Chiller Temperature | −20° | C. |
| Drying Gas Inlet Temperature | 120° | C. |
| Spray Dryer Outlet Temperature (° C.) | 75° | C. |

### PREPARATION 3

### 3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate, SDD preparation

3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate, is dissolved in EtOH, and denatured with MeOH (5% v/v or w/w). A 20% w/w solids solution is prepared with 30% w/w of the solid fraction composed of the title compound; (on a free acid basis) and the balance composed of PVP-VA. This translates to 6% of the title compound (on a free acid basis), 14% PVP-VA and 80% of denatured EtOH SDA-3A—all fractions as w/w. After spray drying, the solids that form are composed of 30% w/w of the solid fraction composed of the title compound (on a free acid basis) and the balance composed of PVP-VA. The % values are shown in Table 2 below.

### Formulation of Preparation 3 SDD

TABLE 2

| | Solution | Amorphous SDD |
|---|---|---|
| | (mass fractions) | (mass fraction) |
| Title compound | 6% | 30.0% |
| PVP-VA | 14% | 70.0% |
| EtOH SDA-3A | 80% | |
| EtOH | 76% | |
| MeOH | 4% | |

Once the solution is prepared, the solution is pumped to a spray dryer where the solution atomizes upon entry. Heated drying gas enters co-current to the atomized liquid at the top of the spray drying chamber at an approximate ratio of 0.044 kg/kg of spray solution to drying gas. The inlet temperature is adjusted to provide an outlet temperature of 35 to 45° C. The solids formed in the spray dryer are collected from a cyclone as well as a filter housing on the gas stream. The gas is passed over a condenser maintained at −3° C. to remove (to a dewpoint of −3° C.) solvent. The gas is then heated to the inlet temperature and passed back to the spray dryer.

### PREPARATION 4

### 3-[(1S,2S)-1-[5-[(4S)-2,2-dimethyloxan-4-yl]-2-[(4S)-2-(4-fluoro-3hou5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate

### Capsule Preparation

Capsules are prepared by first adding sodium bicarbonate (600 mg) to a size 0 hypromellose capsule followed by Preparation 3 SDD (54 mg)

### Quantitative Capsule Composition, Preparation 4

TABLE 3

| Capsule | Wt % | mg/tablet |
|---|---|---|
| Preparation 3 SDD | 8.26 | 54 |
| Sodium bicarbonate (anhydrous) | 91.74 | 600 |
| Total | 100 | 654 |

### PREPARATION 5

### 3-[(1S,2S)-1-[5-[(4S)-2,2-Dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate, tablet formulation

### Core Tablet Composition Preparation

To prepare core tablets, 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate (13.3% w/w) SDD and excipients, (as set forth in Examples 1 through 7), except magnesium stearate, are screened through a 600 μm sieve before use. The materials are added to a vessel (1000 mL) and mixed with a mixer at 32 rpm for about 10 min. The mixture blend is passed through sieve (600 μm) and mixed with mixer a 2^{nd} time at 32 rpm for about 10 min. Magnesium stearate is added and the mixture is mixed at 32 rpm for about 3 min. The mixture blend is compressed using a 14.10×7.75 mm oval tablet tooling on a single station tablet press to produce tablets (400.0 mg) with a hardness of 30 kP.

Tablet compositions are prepared substantially as described herein above, using parameters of the following Tables 4 through 10, to prepare corresponding Examples 1 through 7 (Compositions T1, T2, A, B, C, D, and E). Examples 1 to 7 each include an IR coating substantially as described herein and additionally may include an enteric coating for tablets in Examples 1-6. A tablet of Example 7 includes only an IR coating.

### Example 1 Tablet Composition T1

TABLE 4

| | | Example 1 Composition T1 | | |
|---|---|---|---|---|
| | | | | Quantity/300 |
| Component | Function | % w/w | mg/tablet | g batch (g) |
| Prep 2 SDD (30 wt %) | Drug | 13.3 | 53.3 *(16) | 39.5 (40) |
| Microcrystalline | Filler | 56.7 | 226.7 | 170.5 (170) |
| cellulose | | | | |
| Na₂CO₃ anhydrous | pH modifier | 10 | 40 | 30 |
| Crospovidone XL-10 | disintegrant | 17.5 | 70 | 52.5 |
| Silicon dioxide | Glidant | 2 | 8 | 6 |
| Magnesium stearate | Lubricant | 0.50 | 2.00 | 1.5 |
| Total | | 100 | 400 | 300 |

### Example 2 Tablet Composition T2

TABLE 5

| | | Example 2 Composition T2 | | |
|---|---|---|---|---|
| | | | | Quantity/300 |
| Component | Function | % w/w | mg/tablet | g batch (g) |
| Prep 2 SDD (30 wt %) | Drug | 13.3 | 53.3 (16) | 40 |
| Microcrystalline | Filler | 72.17 | 288.67 | 216.5 |
| cellulose | | | | |
| Na₂CO₃ anhydrous | pH modifier | 10 | 40 | 30 |
| Crospovidone XL-10 | disintegrant | 2 | 8 | 6 |
| Silicon dioxide | Glidant | 2 | 8 | 6 |
| Magnesium stearate | lubricant | 0.5 | 2 | 1.5 |
| Total | | 100 | 400 | 300 |

### Example 3 Tablet Composition A

TABLE 6

| | | Example 3 Composition A | | |
|---|---|---|---|---|
| | | | | Quantity/300 |
| Component | Function | % w/w | mg/tablet | g batch (g) |
| Prep 2 SDD (30 wt %) | Drug | 13.3 | 53.3 (16) | 38.9 |
| Microcrystalline | Filler | 61.7 | 246.7 | 186.1 |
| cellulose | | | | |
| Na₂CO₃ anhydrous | pH modifier | 5 | 20 | 15 |
| Crospovidone XL-10 | disintegrant | 17.5 | 70 | 52.5 |
| Silicon dioxide | Glidant | 2 | 8 | 6 |
| Magnesium stearate | lubricant | 0.5 | 2 | 1.5 |
| Total | | 100 | 400 | 300 |

### Example 4 Tablet Composition B

TABLE 7

| | | Example 4 Composition B | | |
|---|---|---|---|---|
| | | | | Quantity/300 |
| Component | Function | % w/w | mg/tablet | g batch (g) |
| Prep 2 SDD (30 wt %) | Drug | 13.3 | 53.3 (16) | 38.9 |
| Microcrystalline | Filler | 51.7 | 206.7 | 156 |
| cellulose | | | | |
| Na₂CO₃ anhydrous | pH modifier | 15 | 60 | 45 |
| Crospovidone XL-10 | disintegrant | 17.5 | 70 | 52.5 |
| Silicon dioxide | Glidant | 2 | 8 | 6 |
| Magnesium stearate | Lubricant | 0.5 | 2 | 1.5 |
| Total | | 100 | 400 | 300 |

### Example 5 Tablet Composition C

TABLE 8

| | | Example 5 Composition C | | |
|---|---|---|---|---|
| | | | mg/ | Quantity/300 |
| Component | Function | % w/w | tablet | g batch (g) |
| Prep 2 SDD (30 wt %) | Drug | 13.3 | 53.3 (16) | 38.9 |
| Microcrystalline | Filler | 69.2 | 276.7 | 208.6 |
| cellulose | | | | |
| Na₂CO₃ anhydrous | pH modifier | 15 | 60 | 45 |
| Silicon dioxide | Glidant | 2 | 8 | 6 |
| Magnesium stearate | Lubricant | 0.5 | 2 | 1.5 |
| Total | | 100 | 400 | 300 |

### Example 6 Tablet Composition D

TABLE 9

| | | Example 6 Composition D | | |
|---|---|---|---|---|
| | | | mg/ | Quantity/300 |
| Component | Function | % w/w | tablet | g batch (g) |
| Prep 2 SDD (30 wt %) | Drug | 13.3 | 53.3 (16) | 40 |
| Microcrystalline | Filler | 79.2 | 316.7 | 20 |
| cellulose | | | | |
| Na₂CO₃ anhydrous | pH modifier | 5 | 20 | 8 |
| Silicon dioxide | Glidant | 2 | 8 | 2 |
| Magnesium stearate | Lubricant | 0.5 | 2 | 400 |
| Total | | 100 | 400 | 300 |

### Example 7 Tablet Composition E

TABLE 10

| | | Example 7 Composition E | | |
|---|---|---|---|---|
| | | | mg/ | Quantity/300 |
| Component | Function | % w/w | tablet | g batch (g) |
| Prep 2 SDD (30 wt %) | Drug | 13.3 | 53.3 (16) | 38.9 |
| Microcrystalline | Filler | 56.7 | 226.7 | 171 |
| cellulose | | | | |
| Na₂CO₃ anhydrous | pH modifier | 10 | 40 | 30 |
| Crospovidone XL-10 | Disintegrant | 17.5 | 70 | 52.5 |
| Silicon dioxide | Glidant | 2 | 8 | 6 |
| Magnesium stearate | Lubricant | 0.5 | 2 | 1.5 |
| Total | | 100 | 400 | 300 |

*Numbers in ( ) indicate theoretical targets of GLP1RA.

### Immediate Release Coating for Examples 1 Through Example 7

### Immediate Release Tablet

### Coating

A core tablet composition, prepared substantially as described by each of Examples 1 to 7 is film coated to a target 3 wt % gain (with an acceptable range of 2.5 to 3.5 percent) of immediate release coating, such as Opadry® Clear (03K19229) (Colorcon Inc). Tablets are coated using conventional pan coating equipment and vendor recommended coating conditions using water as the base for the spray suspension. Composition Examples 1 to 7 are coated using this immediate release coating.

### Example 8

### Delayed Release Tablet Composition Coating (Enteric Coating)

A tablet composition of Example 1-6 is coated substantially as described above with Opadry® Clear. The Opadry® Clear coated tablets are further enterically coated to a target 6.75 wt % gain (with an acceptable range of 6.25 to 7.25 percent) with Acryl-EZE® (93A18597) (Colorcon Inc) and with PEG 8000 as a plasticizer at 8 wt % relative to Acryl-EZE. Tablets are coated using conventional pan coating equipment with vendor recommended coating conditions using water as the base for the spray suspension.

### Example 9

Enteric Coated Tablet Compositions-Enteric coated tablets are prepared using tablet compositions substantially as described by Examples 1 to 6, except that the wt % of sodium carbonate is adjusted and compensated for by adjusting microcrystalline cellulose content. Tablets are coated with Opadry® 03K19229 (Colorcon Inc.) using 8 wt % solids in the spray suspension to a target weight gain of 3%. Tablets are coated using a benchtop pan coater with a 0.8 mm orifice spray nozzle spraygun with AntiBearding Cap, inlet airflow of 30 cfm, inlet air temperature of 70° C., spray suspension rate of 2.3 g per minute, atomization pressure of 1.5 bar, a pattern air pressure of 0.25 bar and pan speed of 32 rpm. Tablets are then enterically coated to a wt % gain target of approximately 6.5% with Acryl-EZE 93A18597 (Colorcon Inc.) using the same equipment. The aqueous suspension is prepared at 20 wt % Acryl-EZE powder with PEG-8000 added as plasticizer at 8 wt % relative the Acryl-EZE. Coating conditions are as follows: 0.8 mm orifice spray nozzle spraygun with AntiBearding Cap, inlet airflow of 25 cfm, inlet air temperature of 60° C., spray suspension rate of 4.1 g per minute, atomization pressure of 1.5 bar, a pattern air pressure of 0.25 bar and pan speed of 35 rpm.

### Example 10

### Quantitative IR Tablet Composition

TABLE 11

| IR Tablet | Wt % | mg/tablet |
|---|---|---|
| Preparation 3 SDD | 13.3 | 53.3 |
| Microcrystalline cellulose 102 | 64.2 | 256.7 |
| Sodium carbonate (anhydrous) | 15 | 60 |
| Crospovidone | 5 | 20 |
| Magnesium Stearate | 0.5 | 2 |
| Silicon Dioxide | 2 | 8 |
| Total | 100 | 400 |

### Example 11

### Alternate Tablet Composition Super Disintegrant with Croscarmellose Sodium

TABLE 12

| | Wt % | mg/tablet |
|---|---|---|
| Preparation 3 SDD | 13.9 | 55.8 |
| Microcrystalline Cellulose 102 | 58 | 232 |
| Sodium carbonate (anhydrous) | 20 | 80 |
| Croscarmellose Sodium | 5 | 20 |
| Magnesium Stearate | 1 | 4 |
| Silicon Dioxide | 2 | 8 |
| Total | 100 | 400 |

### Example 12

### Alternate Tablet Composition Super Disintegrant with Crospovidone

TABLE 13

| | Wt % | mg/tablet |
|---|---|---|
| Preparation 3 SDD | 13.8 | 55.8 |
| Silicified microcrystalline cellulose | 57 | 232 |
| Sodium carbonate (anhydrous | 20 | 80 |
| Crospovidone | 6.2 | 25 |
| Magnesium Stearate | 1 | 4 |
| Silicon Dioxide | 2 | 8 |
| Total | 100 | 405 |

Tablet compositions are prepared with these super disintegrants in the formulas as shown in Tables 11 and 12. All materials are added, excluding magnesium stearate, to an amber glass jar and mixed using a rotational diffusional mixer at 22 rpm for 10 minutes. The blend is passed through a #35 mesh screen and then mixed for another 10 minutes. The magnesium stearate is passed through a #35 mesh and added to the jar and blended for 5 minutes. The blend is compressed using a single station tablet press at 6 kN compression force using modified oval tooling. Core tablets are coated with Opadry® 03K19229 (Colorcon Inc.) using 8 wt % solids in the spray suspension to a weight gain target of 3%. Tablets are coated using a benchtop pan coater with a 0.8 mm orifice spray nozzle spraygun with AntiBearding Cap, inlet airflow of 30 cfm, inlet air temperature of 70° C., spray suspension rate of 2.3 g per minute, atomization pressure of 1.5 bar, a pattern air pressure of 0.25 bar and pan speed of 32 rpm. Tablets are then enterically coated to a wt % gain target of 6.5% with Acryl-Eze 93A18597 (Colorcon Inc.) using the same equipment. The aqueous suspension is prepared at 20 wt % Acryl-EZE powder with PEG-8000 added as plasticizer at 8 wt % relative the Acryl-EZE. Coating conditions are as follows: 0.8 mm orifice spray nozzle spraygun with AntiBearding Cap, inlet airflow of 25 cfm, inlet air temperature of 60° C., spray suspension rate of 4.1 g per minute, atomization pressure of 1.5 bar, a pattern air pressure of 0.25 bar and pan speed of 35 rpm.

### Example 13

### Tablet Composition 400 mg Total Weight Tablet

Total weight tablets (400 mg) are prepared using the percentages in Table 14. Tablets are prepared, coated with the immediate release coating, and then the enteric coating substantially as described by Example 9.

TABLE 14

| | Wt % in a 400 mg total tablet weight | | | | | |
|---|---|---|---|---|---|---|
| | Example | Example | Example | Example | Example | Example |
| | 1; T1 | 2; T2 | 3; A | 4; B | 5; C | 6; D |
| Preparation 3 SDD | 13.3 | 13.3 | 13.3 | 13.3 | 13.3 | 13.3 |
| Microcrystalline | 57 | 72 | 62 | 52 | 69 | 79 |
| cellulose 102 | | | | | | |
| Sodium carbonate | 10 | 10 | 5 | 15 | 15 | 5 |
| (anhydrous) | | | | | | |
| Crospovidone | 17.5 | 2 | 17.5 | 17.5 | 0 | 0 |
| Silixon Dioxide | 2 | 2 | 2 | 2 | 2 | 2 |
| Magnesium stearate | 0.5 | 0.5 | 0.5 | 0.5 | 0.5 | 0.5 |
| Total | 100 | 100 | 100 | 100 | 100 | 100 |

### Example 14

### Tablet Compositions 9-30

Core tablet compositions 9-30 (Comp. 9-Comp. 30) are prepared using the percentages in the following table and processes as described earlier. Core tablets are prepared, coated with the immediate release coating, and optionally, can be further coated with the enteric coating substantially as described in Example 9.

| | Comp. 9 | Comp. 10 | Comp. 11 | Comp. 12 |
|---|---|---|---|---|
| | 75 mg | 60 mg | 45 mg | 36 mg |
| | GLP1RA | GLP1RA | GLP1RA | GLP1RA |
| | (wt % of | (wt % of | (wt % of | (wt % of |
| Ingredient | composition) | composition) | composition) | composition) |
| Prep 2 SDD | 250 | 200 | 150 | 120 |
| (30 wt % of GLP1RA) | (25%) | (25%) | (21%) | (19.5%) |
| Microcrystalline | 495 | 396 | 382 | 338 |
| cellulose | (49.5%) | (49.5%) | (53.5%) | (55%) |
| Crospovidone XL-10 | 170 | 136 | 121 | 104 |
| | (17%) | (17%) | (17%) | (17%) |
| Na₂CO₃ anhydrous | 80 | 64 | 57 | 49 |
| | (8%) | (8%) | (8%) | (8%) |
| Magnesium stearate | 5 | 4 | 3.6 | 3.1 |
| | (0.5%) | (0.5%) | (0.5%) | (0.5%) |
| Total | 1000 mg | 800 mg | 714 mg | 615 mg |
| | Comp. 13 | Comp. 14 | Comp. 15 | Comp. 16 |
| | 30 mg | 25.7 mg | 24 mg | 20 mg |
| | GLP1RA | GLP1RA | GLP1RA | GLP1RA |
| | (wt % of | (wt % of | (wt % of | (wt % of |
| Ingredient | composition) | composition) | composition) | composition) |
| Prep 2 SDD | 100 | 85.7 | 80 | 67 |
| (30 wt % of GLP1RA) | (19.5%) | (19.5%) | (19.5%) | (19.5%) |
| Microcrystalline | 282 | 242 | 225 | 188 |
| cellulose | (55%) | (55%) | (55%) | (55%) |
| Crospovidone XL-10 | 87 | 75 | 70 | 58 |
| | (17%) | (17%) | (17%) | (17%) |
| Na₂CO₃ anhydrous | 41 | 35 | 33 | 27 |
| | (8%) | (8%) | (8%) | (8%) |
| Magnesium stearate | 2.6 | 2.2 | 2.1 | 1.7 |
| | (0.5%) | (0.5%) | (0.5%) | (0.5%) |
| Total | 513 mg | 440 mg | 410 mg | 342 mg |
| | Comp. 17 | Comp. 18 | Comp. 19 | Comp. 20 |
| | 17 mg | 12 mg | 10 mg | 8.6 mg |
| | GLP1RA | GLP1RA | GLP1RA | GLP1RA |
| | (wt % of | (wt % of | (wt % of | (wt % of |
| Ingredient | composition) | composition) | composition) | composition) |
| Prep 2 SDD | 57 | 40 | 33 | 29 |
| (30 wt % of GLP1RA) | (19.5%) | (19.5%) | (19.5%) | (19.5%) |
| Microcrystalline | 161 | 113 | 94 | 81 |
| cellulose | (55%) | (55%) | (55%) | (55%) |
| Crospovidone XL-10 | 50 | 35 | 29 | 25 |
| | (17%) | (17%) | (17%) | (17%) |
| Na₂CO₃ anhydrous | 23 | 16 | 14 | 12 |
| | (8%) | (8%) | (8%) | (8%) |
| Magnesium stearate | 1.5 | 1.0 | 0.85 | 0.73 |
| | (0.5%) | (0.5%) | (0.5%) | (0.5%) |
| Total | 293 mg | 205 mg | 171 mg | 147 mg |
| | Comp. 21 | Comp. 22 | Comp. 23 | Comp. 24 |
| | 6 mg | 5 mg | 4.3 mg | 3 mg |
| | GLP1RA | GLP1RA | GLP1RA | GLP1RA |
| | (wt % of | (wt % of | (wt % of | (wt % of |
| Ingredient | composition) | composition) | composition) | composition) |
| Prep 2 SDD | 20 | 17 | 14.3 | 10 |
| (30 wt % of GLP1RA) | (19.5%) | (19.5%) | (16.9%) | (11.8%) |
| Microcrystalline | 56 | 47 | 55 | 59 |
| cellulose | (55%) | (55%) | (65%) | (70%) |
| Crospovidone XL-10 | 17 | 10 | 8.5 | 8.5 |
| | (17%) | (17%) | (10%) | (10%) |
| Na₂CO₃ anhydrous | 8.2 | 6.8 | 6.8 | 6.8 |
| | (8%) | (8%) | (8%) | (8%) |
| Magnesium stearate | 0.51 | 0.43 | 0.43 | 0.43 |
| | (0.5%) | (0.5%) | (0.5%) | (0.5%) |
| Total | 103 mg | 100 mg | 100 mg | 85 mg |
| | Comp. 25 | Comp. 26 | Comp. 27 | Comp. 28 |
| | 2.5 mg | 2.1 mg | 1.0 mg | 0.8 mg |
| | GLP1RA | GLP1RA | GLP1RA | GLP1RA |
| | (wt % of | (wt % of | (wt % of | (wt % of |
| Ingredient | composition) | composition) | composition) | composition) |
| Prep 2 SDD | 8.3 | 7 | 3.3 | 2.7 |
| (30 wt % of GLP1RA) | (9.8%) | (8.2%) | (3.9%) | (3.2%) |
| Microcrystalline | 61 | 62 | 66 | 67 |
| cellulose | (72%) | (73%) | (78%) | (78%) |
| Crospovidone XL-10 | 8.5 | 8.5 | 8.5 | 8.5 |
| | (10%) | (10%) | (10%) | (10%) |
| Na₂CO₃ anhydrous | 6.8 | 6.8 | 6.8 | 6.8 |
| | (8%) | (8%) | (8%) | (8%) |
| Magnesium stearate | 0.43 | 0.43 | 0.43 | 0.43 |
| | (0.5%) | (0.5%) | (0.5%) | (0.5%) |
| Total | 85 mg | 85 mg | 85 mg | 85 mg |

| | Comp. 29 | Comp. 30 |
|---|---|---|
| | 0.7 mg | 0.5 mg |
| | GLP1RA | GLP1RA |
| | (wt % of | (wt % of |
| Ingredient | composition) | composition) |
| Prep 2 SDD | 2.3 | 1.7 |
| (30 wt % of GLP1RA) | (2.7%) | (2.0%) |
| Microcrystalline | 67 | 68 |
| cellulose | (79%) | (80%) |
| Crospovidone XL-10 | 8.5 | 8.5 |
| | (10%) | (10%) |
| Na₂CO₃ anhydrous | 6.8 | 6.8 |
| | (8%) | (8%) |
| Magnesium stearate | 0.43 | 0.43 |
| | (0.5%) | (0.5%) |
| Total | 85 mg | 85 mg |

### Particle Size Measurement

Particle size of the SDD was determined by laser diffraction utilizing wet dispersion on the Malvern Mastersizer 3000 equipped with the Hydro MV (medium volume) liquid disperser (Malvern Instruments Ltd. UK.). Optical model: Mie model, obscuration limits: 5-30%, general purpose model. The volume-based distribution was measured and the (D10, D50, D90) quantiles were reported.

The SDDs of Preparations 2 and 3 have a mean particle size of about 40 to about 65 μm, or more specifically, about 40 to about 50 μm, in diameter, as measured by the above method.

### Solubility Studies

Twenty-four Hour Aqueous Solubility of Crystalline GLP1RA-Ca at 25° C.

TABLE 15

| | | Solubility at 25° C. |
|---|---|---|
| Solvent | Media pH | (mg/mL) |
| Water | NM* | 0.028 |
| 0.1N HCl | 1.01 | <0.001 |
| 0.01N HCl | 1.98 | <0.001 |
| pH 4.5 acetate buffer (USP) | 4.5 | <0.001 |
| pH 6.0 phosphate buffer (USP) | 5.99 | <0.001 |
| pH 7.5 phosphate buffer (USP) | 7.44 | 0.003 |
| Simulated gastric fluid | 1.97 | 0.003 |
| Fasted state simulated intestinal fluid | 6.49 | 0.002 |
| Fed state simulated intestinal fluid | 4.93 | 0.013 |

*Not Measured

An embodiment of the present invention is an oral tablet formulation comprising an amorphous SDD of GLP1RA-Ca. GLP1RA-Ca in its crystalline form has poor aqueous solubility as shown in Table 15. An amorphous SDD of GLP1RA-Ca using PVP-VA as the carrier polymer has been shown to provide improved hydrophilicity and solubility as compared to the crystalline GLP1RA-Ca (Table 16).

### Twenty-Four Hour Aqueous Solubility of Preparation 3 SDD at 25° C.

TABLE 16

| | | Solubility at 25° C. |
|---|---|---|
| Solvent | Media pH | (mg/mL) |
| Water | 6.9 | 90.0 |
| 0.1N HCl | 1.0 | 0.005 |
| 0.01N HCl | 2.0 | 0.004 |
| pH 4.5 acetate buffer (USP) | 4.5 | 0.007 |
| pH 6.0 phosphate buffer (USP) | 6.0 | 0.100 |
| pH 7.5 phosphate buffer (USP) | 7.5 | 8.7 |
| Simulated gastric fluid | 2.0 | 0.110 |
| Fasted state simulated intestinal fluid | 6.5 | 0.620 |
| Fed state simulated intestinal fluid | 5.0 | 0.022 |

### Twenty-Four-Hour Aqueous Solubility of the Preparation 3, SDD at 37° C.

TABLE 17

| | | 24 hr solubility at 37° C. |
|---|---|---|
| Solvent | pH | (mg/mL) |
| Water | 6.9 | 86.5 |
| 0.1N HCl | 1.0 | 0.001 |
| 0.01N HCl | 2.0 | 0.001 |
| pH 4.5 acetate buffer (USP) | 4.5 | 0.001 |
| pH 6.0 phosphate buffer (USP) | 6.0 | 0.038 |
| pH 7.5 phosphate buffer (USP) | 7.5 | 8.4 |
| Simulated gastric fluid | 2.0 | 0.044 |
| Fasted state simulated intestinal fluid | 6.5 | 0.005 |
| Fed state simulated intestinal fluid | 5.0 | 0.003 |

As seen in Tables 16 and 17, Preparation 3 has pH dependent solubility. With a pKa of 5.1, it is expected that solubility of the drug would begin to increase at pH values near the pKa and dissolution performance will improve at pH values at or above the pKa. To test this, capsules containing the SDD and sufficient amounts of sodium bicarbonate to raise the pH of an acidic dissolution media to around the pKa were evaluated in dissolution experiments compared to a tablet containing much less pH modifier. The quantitative formulations are shown in Table 11 for the tablet and Table 3 for the capsule.

### Dosage Dissolution Studies

Dosage forms are evaluated in a 2-stage dissolution test consisting of an acid stage (500 mL 0.0133N HCl/8.12 mM NaCl) for 1 hour followed by the addition of concentrated FaSSIF (400 mL) to convert media to the same pH and composition of conventional fasted state intestinal fluid. To prepare concentrated FaSSiF for each 1 L, sodium phosphate dibasic anhydrous (5.714 g), sodium phosphate monobasic anhydrous (2.906 g), and NaCl (12.354 g) are added together with water to make 1 L and mixed well. The pH should be about 7.0. Add SIF powder (5.6 g) (FaSSIF/FeSSIF/FaSSGF powder (Biorelevant)).

The test is run at 100 rpm using a USP II dissolution apparatus. Aliquots are withdrawn, filtered through a 0.22 micron filter, diluted 1:1 with MeOH to prevent drug precipitation and analyzed by HPLC.

It was surprisingly found that the IR tablet performed better in the gastric stage than the capsule formulation, despite the capsule formulation raising the pH in the media to above 5 due to the large amount of sodium bicarbonate and the tablet having no effect on the media pH, which remained at a pH of 2.

### Dissolution Comparisons of Example 10 Tablet and Preparation 4 Capsule

TABLE 18

| | | % Dissolved | | | | | | |
|---|---|---|---|---|---|---|---|---|
| | | 0.0133N HCl/8.12 mM NaCl | | | Fasted state intestinal fluid | | | |
| | | 0.25 hr | 0.5 hr | 1 hr | 2 hr | 3 hr | 5 hr | 5.5 hr |
| Example | R1 | 40.7225 | 34.7522 | 23.9687 | 8.9237 | 5.5276 | 6.8771 | 5.6406 |
| 10 IR | R2 | 36.3592 | 33.8439 | 24.5525 | 11.3698 | 5.8262 | 5.6113 | 4.7865 |
| Tablet | R3 | 35.577 | 38.8444 | 32.1674 | 13.9839 | 8.9858 | 6.6275 | 7.5356 |
| | Ave | 37.5529 | 35.8135 | 26.8962 | 11.4258 | 6.779867 | 6.371967 | 5.987567 |
| | StDev | 2.772676 | 2.663835 | 4.574316 | 2.530565 | 1.916219 | 0.670474 | 1.40701 |
| | RSD | 7.383387 | 7.438076 | 17.0073 | 22.14781 | 28.26338 | 10.52225 | 23.49886 |
| Preparation | R1 | 7.0103 | 6.5912 | 5.9243 | 8.1787 | 10.0586 | 11.0423 | 15.85 |
| 4 Capsule | R2 | 0 | 0 | 4.0373 | 6.8174 | 7.3107 | 8.0334 | 9.687 |
| | R3 | 9.2946 | 11.4677 | 9.5824 | 11.7023 | 15.0912 | 13.2156 | 15.5464 |
| | Ave | 5.434967 | 6.019633 | 6.514667 | 8.899467 | 10.82017 | 10.76377 | 13.69447 |
| | StDev | 4.843413 | 5.755176 | 2.819297 | 2.52095 | 3.945761 | 2.602304 | 3.473886 |
| | RSD | 89.11579 | 95.60676 | 43.27614 | 28.32698 | 36.46673 | 24.17652 | 25.36708 |

StDev refers to standard deviation.

Capsule Preparation 4 and IR tablet composition Example 7 are also compared using alternate dissolution conditions. Conditions used are USP II paddle at 100 rpm with 50 mM pH 6.8 phosphate buffer with 2% sodium lauryl sulfate as the dissolution media. Samples are analyzed by HPLC.

Surprisingly, despite having a significant amount of sodium bicarbonate present that should facilitate dissolution of the SDD from the capsule, the IR tablet again released more rapidly than the capsule with 100% release achieved within 30 minutes, while the capsule only achieved 35% release at 30 minutes and did not achieve >90% release until about 75 minutes.

### PREPARATION 4 Capsule Alternate Dissolution Conditions

TABLE 19

| Time (minutes) | % released |
|---|---|
| 10 | 0.40 |
| 20 | 14.20 |
| 30 | 35.20 |
| 45 | 71.80 |
| 60 | 87.00 |
| 75 | 94.80 |
| 90 | 96.20 |
| 120 | 96.60 |

### Composition Example 7 Alternate Dissolution Conditions

TABLE 20

| Time (minutes) | % released |
|---|---|
| 15 | 90.90 |
| 30 | 100.50 |
| 45 | 100.70 |
| 60 | 100.70 |
| 90 | 100.80 |
| 120 | 100.80 |
| 150 | 100.80 |

The pH modifiers in both the IR and DR tablets facilitate dissolution of the weakly acidic drug by raising the local, or micro, pH of the tablet as it disintegrates. Enteric tablets are used to discover levels of pH modifier required for enteric tablet compositions. Enteric Coated Tablets, Example 9, are tested in a 2-stage dissolution test using a USP II apparatus with paddle speed of 100 rpm. An acid challenge stage is conducted first lasting 2 hours in pH 4.5 sodium acetate buffer (500 mL) after which the tablets are removed and placed into FaSSiF (Biorelevant) (500 mL). Aliquots are withdrawn, filtered through a 0.22 micron filter, diluted 1:1 with MeOH to prevent drug precipitation and analyzed by HPLC.

Unexpectedly, very high levels of pH modifier (20 wt % in the tablet) resulted in poorer dissolution behavior of the DR tablets compared to lower levels. Further, an optimal level of peak and more sustained dissolution is achieved at around 10 wt % sodium carbonate.

### Dissolution Studies for Delayed Release Tablet

TABLE 21

| | | | % Dissolved in Simulated | | | | | | |
|---|---|---|---|---|---|---|---|---|---|
| Example | % | | fasted intestinal fluid | | | | | | |
| # | Na₂CO₃ | Replicate | 0.5 hr | 1 hr | 2 hr | 3 hr | 4 hr | 5 hr | 5.5 hr |
| Example | 0 | R1 | 5.7 | 10.0 | 12.3 | 10.0 | 6.6 | 5.8 | 7.0 |
| 9a | | R2 | 3.5 | 8.3 | 12.6 | 10.4 | 7.0 | 6.5 | 7.0 |
| | | R3 | 4.3 | 7.9 | 13.7 | 11.1 | 6.7 | 5.5 | 5.8 |
| | | Ave | 4.5 | 8.7 | 12.9 | 10.5 | 6.8 | 6.0 | 6.6 |
| | | StDev | 1.1 | 1.1 | 0.7 | 0.6 | 0.2 | 0.5 | 0.7 |
| | | RSD | 24.8 | 12.6 | 5.5 | 5.5 | 3.2 | 8.8 | 10.3 |
| Example | 5 | R1 | 62.0 | 60.2 | 47.6 | 15.1 | 7.1 | 5.2 | 4.9 |
| 9b | | R2 | 55.8 | 58.8 | 53.8 | 34.5 | 10.8 | 6.8 | 6.7 |
| | | R3 | 57.6 | 58.9 | 48.4 | 21.8 | 8.2 | 5.8 | 5.8 |
| | | Ave | 58.5 | 59.3 | 49.9 | 23.8 | 8.7 | 5.9 | 5.8 |
| | | StDev | 3.2 | 0.8 | 3.4 | 9.9 | 1.9 | 0.8 | 0.9 |
| | | RSD | 5.5 | 1.3 | 6.7 | 41.6 | 21.6 | 13.6 | 15.2 |
| Example | 10 | R1 | 43.9 | 64.4 | 73.6 | 65.9 | 44.9 | 16.1 | 12.0 |
| 9c | | R2 | 49.2 | 69.2 | 76.1 | 72.9 | 61.8 | 34.7 | 20.0 |
| | | R3 | 50.9 | 70.1 | 75.8 | 67.3 | 43.9 | 14.1 | 13.1 |
| | | Ave | 48.0 | 67.9 | 75.2 | 68.7 | 50.2 | 21.6 | 15.0 |
| | | StDev | 3.6 | 3.0 | 1.4 | 3.7 | 10.1 | 11.4 | 4.3 |
| | | RSD | 7.6 | 4.5 | 1.8 | 5.4 | 20.1 | 52.6 | 28.6 |
| Example | 15 | R1 | 57.6 | 56.8 | 51.5 | 23.3 | nm | 5.6 | 4.9 |
| 9d | | R2 | 57.2 | 55.3 | 58.3 | 30.5 | nm | 6.2 | 6.5 |
| | | R3 | 66.2 | 70.6 | 58.6 | 24.0 | nm | 5.4 | 4.2 |
| | | Ave | 60.3 | 60.9 | 56.1 | 25.9 | nm | 5.7 | 5.2 |
| | | StDev | 5.1 | 8.4 | 4.0 | 4.0 | nm | 0.4 | 1.2 |
| | | RSD | 8.4 | 13.8 | 7.1 | 15.3 | nm | 7.4 | 22.6 |
| Example | 20 | R1 | 25.4 | 42.4 | 57.9 | 28.0 | 9.6 | 7.1 | 5.7 |
| 9e | | R2 | 22.4 | 41.7 | 60.8 | 34.5 | 12.2 | 7.5 | 7.0 |
| | | R3 | 30.3 | 44.3 | 60.3 | 27.3 | 10.0 | 6.8 | 6.5 |
| | | Ave | 26.1 | 42.8 | 59.7 | 29.9 | 10.6 | 7.1 | 6.4 |
| | | StDev | 4.0 | 1.3 | 1.6 | 4.0 | 1.4 | 0.3 | 0.7 |
| | | RSD | 15.3 | 3.1 | 2.6 | 13.3 | 13.2 | 4.4 | 10.3 |

nm = not measured

Tablets of Example 11 and Example 12 are tested in a 2-stage dissolution test using a USP II: apparatus with paddle speed of 100 rpm. The acid challenge stage is 2 hours in pH 4.5 sodium acetate buffer (500 mL) after which the tablets are removed and placed into FaSSiF (Biorelevant) (500 mL). Aliquots are withdrawn, filtered through a 0.22 micron filter, diluted 1:1 with MeOH to prevent drug precipitation and analyzed by HPLC.

It was surprisingly found that croscarmellose sodium as used in Example 11 is not able to afford the desired release profile whereas crospovidone as used in Example 12 affords the desired release profile.

### Two Stage Dissolution Testing of Tablets

TABLE 22

| | | % Dissolved in Simulated fasted intestinal fluid | | | | | |
|---|---|---|---|---|---|---|---|
| | | 0.5 hr | 1 hr | 2 hr | 3 hr | 5 hr | 5.5 hr |
| Croscarmellose | R1 | 4.0 | 23.3 | 64.6 | 78.1 | 79.9 | 83.1 |
| Sodium | R2 | 4.1 | 12.7 | 79.4 | 70.4 | 85.2 | 82.5 |
| Example 11 | R3 | 7.0 | 18.7 | 69.3 | 77.5 | 84.7 | 80.8 |
| | Ave | 5.0 | 18.2 | 71.1 | 75.3 | 83.3 | 82.2 |
| | RSD | 34.5 | 29.2 | 10.6 | 5.6 | 3.5 | 1.4 |
| Crospovidone | R1 | 31.9 | 68.6 | 80.0 | 82.1 | 80.6 | 82.0 |
| Tablet, | R2 | 19.2 | 73.5 | 81.7 | 81.3 | 74.8 | 70.9 |
| Example 12 | R3 | 23.1 | 76.4 | 80.4 | 86.0 | 69.5 | 57.5 |
| | Ave | 24.8 | 72.8 | 80.7 | 83.2 | 75.0 | 70.1 |
| | RSD | 26.4 | 5.4 | 1.1 | 3.1 | 7.4 | 17.5 |

In a further embodiment, the rate of release from the enterically coated tablets may be controlled to affect specific aspects of the PK profile, such as Cmax or the peak to trough plasma concentrations as these may be beneficial to the patient to help with tolerability and/or efficacy.

Controlled release from tablets is often achieved using rate controlling polymers such as hypromellose as in hydrophilic sustained release matrix tablets. The challenge with hypromellose or similar polymers is that they tend to work over several hours through diffusional and/or erosional mechanisms. This time span may be too long for low permeability molecules, where not maximizing drug concentration in the intestinal lumen, if the drug is released gradually over many hours, may result in decreased flux across the intestinal epithelium and subsequently lower absorption.

It was surprising found that controlling the amount of super disintegrant in the enterically coated dosage forms enables a significant degree of control of the rate of release over the desired time of complete release within 30 minutes

Tablets T1 and T2 are evaluated in a 2-stage dissolution test substantially similar to what has already been described, except the pH 4.5 sodium acetate acid stage is set to 1 hour followed by transfer of the tablet to FaSSiF (Biorelevant). The results are shown in Table 23.

Surprisingly, simply modulating the disintegrant levels can achieve a controlled level of release in the FaSSiF.

### Two Stage Dissolution Study of Tablets T1 and T2 at pH 4.5

TABLE 23

| | | Percent dissolved | |
|---|---|---|---|
| Media | Time (min) | Example 1; T1 | Example 2; T2 |
| pH 4.5 sodium | 30 | 0.0 | 0.0 |
| acetate buffer | 60 | 0.0 | 0.0 |
| Fasted state | 90 | 82.2 | 2.9 |
| intestinal fluid | 120 | 85.5 | 9.8 |
| | 180 | 86.0 | 66.1 |
| | 240 | 84.8 | 81.2 |
| | 300 | 76.3 | 69.8 |
| | 330 | 67.6 | 58.0 |

Another lever to modulate the release of the drug from the enteric tablets is to alter the amount of sodium carbonate. The tablets are subjected to dissolution in pH 6.8 phosphate buffer with 2% sodium lauryl sulfate as described herein. Results of this study demonstrate that there is a synergy between the level of disintegrant and the amount of pH modifier that can be used to control release of drug from the tablet. Further, the release is controlled to within the desired range of within 30 minutes to not more than 3 hours to model the approximate in-vivo transit time through the small intestine where most drug absorption is expected to occur. The results are shown in Table 24.

### Dissolution Time Study of Composition Tablets, T1, T2, a, B, C, D at pH 6.8

TABLE 24

| | Percent dissolved in pH 6.8 phospahte | | | | | |
|---|---|---|---|---|---|---|
| | buffer with 2% sodium lauryl sulfate | | | | | |
| | Exam- | Exam- | Exam- | Exam- | Exam- | Exam- |
| Time | ple | ple | ple | ple | ple | ple |
| (min) | 1; T1 | 2; T2 | 3; A | 4; B | 5; C | 6; D |
| 15 | 94.4 | 8.0 | 89.9 | 58.9 | 2.2 | 1.5 |
| 30 | 97.4 | 25.1 | 99.4 | 88.5 | 5.5 | 9.4 |
| 45 | 97.5 | 44.9 | 99.6 | 95.0 | 14.2 | 24.8 |
| 60 | 97.4 | 75.7 | 99.6 | 98.3 | 25.5 | 53.6 |
| 90 | 97.4 | 99.1 | 99.5 | 98.5 | 56.5 | 83.0 |
| 120 | 97.5 | 99.3 | 99.5 | 98.6 | 87.2 | 90.8 |
| 150 | 97.5 | 99.2 | 99.4 | 98.5 | 99.6 | 97.5 |

### Clinical Trial

A multiple-ascending dose study was conducted to characterize and compare the pharmacokinetics (PK) of prototype formulations of Preparation 4 (capsule), Example 1 (DR Tablet composition T1 with enteric coating prepared as in Example 9, referred to below as T1 DR Tablet), and Example 7 (IR Tablet Composition E with immediate release coating, referred to below as E IR Tablet). The study was conducted in two parts: A and B.

Part A evaluated the safety, tolerability, and PK of multiple oral doses of GLP1RA formulation prototypes T1 and E in healthy participants versus the reference capsule Preparation 4. A dose titration period was from Day 1 through Day 18, where participants received increasing doses of GLP1RA in a capsule for dose titration. Within-treatment dose escalation was every 6 days and reached a maximum dose of 16 mg of GLP1RA once daily (QD) on Day 19. On Days 19 to 24, participants received reference capsule Preparation 4 (16 mg QD) before entering the test phase, from Days 25 to 36. During test period-1, the participants were administered GLP1RA a prototype tablet (16 mg QD) formulations as per their randomization. The participants were then crossed over on Day 31 for period-2 and received the second prototype tablet (16 mg QD) through Day 36.

TABLE 25
Geometric Mean (Geometric CV %) Plasma Pharmacokinetic Parameters
for GLP1RA Following Single and Multiple Oral Doses of
GLP1RA to Healthy Male and Female Participants

| | Dose Period | | | | | |
|---|---|---|---|---|---|---|
| | Dose Level | | | | | |
| | Status | | | | | |
| | No. of Participants | | | | | |
| | Reference Period- | | Test Period 1 and | | Test Period 1 and | |
| | Preparation 4 Capsule | | 2- T1, DR tablet | | 2- E, IR tablet | |
| | Fasted | | Fasted | | Fasted | |
| Parameter | n = 10 | | n = 10 | | n = 10 | |
| Single (1^{st}) Dose | 19 | | 25 or 31 | | 25 or 31 | |
| Day | | | | | | |
| Tmax^{a} (h) | 8.00 | (4.00-23.87) | 8.00 | (4.00-23.97) | 8.00 | (4.00-12.05) |
| Cmax (ng/mL) | 59.7 | (64.5) | 89.7 | (80.0) | 82.5 | (86.8) |
| AUC(0-24) | 869 | (51.7) | 1230 | (72.3) | 1180 | (64.3) |
| (ng · h/mL) | | | | | | |
| Multiple (6^{th}) Dose | 24 | | 30 or 36 | | 30 or 36 | |
| Day | | | | | | |
| Tmax^{a} (h) | 7.02 | (4.00-8.00) | 8.00 | (4.00-16.00) | 8.00 | (4.00-16.10) |
| Cmax (ng/mL) | 66.2 | (77.4) | 111 | (81.2) | 97.4 | (58.9) |
| AUC(0-24) | 988 | (66.9) | 1480 | (75.2) | 1400 | (51.2) |

| (ng · h/mL) | | | | | |
|---|---|---|---|---|---|
| Frel Cmax (%) | N/A | 167 | (38.8) | 147 | (36.6) |
| Frel AUC(0-24) (%) | N/A | 150 | (25.0) | 141 | (23.8) |

Abbreviations: AUC(0-24) = area under the curve from time 0 to 24 hours post dose; Cmax = maximum observed concentration; Frel AUC(0-24) = relative bioavailability based on AUC(0-24); Frel Cmax = relative bioavailability based on Cmax; h = hour; N/A = not applicable

The results of Part A indicate that the average peak and overall exposure levels (as measured by Cmax and AUC(0-24)) for 16 mg, DR Tablet T1 were approximately 67% and 50% higher, respectively, than those for 16 mg reference Preparation 4 capsule. These increases were statistically significant at the 10% significance level (p<0.001 for both Cmax and AUC(0-24)).

Similarly, for 16 mg Tablet E IR tablet, the average peak and overall exposure levels (as measured by Cmax and AUC(0-24)) were approximately 47% and 41% higher, respectively, than those for 16 mg reference capsule. These increases were statistically significant at the 10% significance level (p=0.007 and p<. 001 for Cmax and AUC(0-24), respectively).

Part B further evaluated Tablet E IR tablet regarding food effect and DDI effect with a co-administered proton pump inhibitor (PPI). As in Part A, treatment escalation occurred every 6 days during a titration period and reached a maximum dose of 16 mg QD on Day 19. Treatment then proceeded with GLP1RA in a fasted or fed state and a fasted state with a co-administered PPI. Each option began with a 6-day reference period (16 mg QD) using Tablet E IR tablet as the reference, followed by two 6-day test periods (16 mg QD).

TABLE 26
Geometric Mean (Geometric CV %) Plasma Pharmacokinetic Parameters for GLP1RA Following
Single and Multiple Oral Doses of GLP1RA to Healthy Male and Female Participants

| | Dose Period | | | | | |
|---|---|---|---|---|---|---|
| | Dose Level | | | | | |
| | Status | | | | | |
| | No. of Participants | | | | | |
| | Reference Period | | Test Period 1 | | Test Period 2 | |
| | Tablet E IR tablet | | Tablet E IR tablet | | Tablet E IR tablet + PPI | |
| | Fasted | | Fed | | Fasted | |
| Parameter | n = 12 | | n = 12 | | n = 12 | |
| Single (1^{st}) Dose Day | 19 | | 25 | | 31 | |
| Tmax^{a} (h) | 6.00 | (4.00-8.02) | 8.02 (4.02-12.02) [n = 11] | | 6.00 (4.00-16.02) [n = 11] | |
| Cmax (ng/mL) | 63.8 | (33.5) | 48.0 (35.2) [n = 11] | | 74.7 (27.1) [n = 11] | |
| AUC(0-24) (ng · h/mL) | 869 | (27.1) | 781 (31.2) [n = 11] | | 1080 (22.7) [n = 11] | |
| Multiple (6^{th}) Dose Day | 24 | | 30 | | 36 | |
| Tmax^{a} (h) | 8.00 | (4.00-12.00) | 7.00 | (2.00-16.05) | 7.00 | (4.00-12.00) |
| Cmax (ng/mL) | 63.4 | (32.2) | 56.3 | (29.6) | 59.6 | (51.0) |
| AUC(0-24) (ng · h/mL) | 903 | (25.3) | 865 | (24.6) | 956 | (40.1) |

| | | | | | |
|---|---|---|---|---|---|
| Frel Cmax (%) | N/A | 88.9 | (38.1) | 94.1 | (36.3) |
| Frel AUC(0-24) (%) | N/A | 95.8 | (23.8) | 106 | (31.6) |

Abbreviations: AUC(0-24) = area under the curve from time 0 to 24 hours post dose; Cmax = maximum observed concentration; Frel AUC(0-24) = relative bioavailability based on AUC(0-24); Frel Cmax = relative bioavailability based on Cmax; h = hour; N/A = not applicable

The results of Part B indicate that the average peak and overall exposure levels (as measured by Cmax and AUC(0-24)) for 16 mg Tablet E IR tablet fed were comparable (that is, fed exposure within 11% and 4% of fasted exposure for Cmax and AUC(0-24), respectively) to those for 16 mg Tablet E IR tablet fasted (p=0.39 and p=0.66 for Cmax and AUC(0-24), respectively).

For 16 mg Tablet E IR tablet with PPI, the average peak and overall exposure levels (as measured by Cmax and AUC(0-24)) were also comparable (that is, 6% lower and 6% higher for Cmax and AUC(0-24), respectively) to those for 16 mg IR Tablet Composition E (p=0.65 and p=0.57 for Cmax and AUC(0-24), respectively).

Although the invention has been described in considerable detail with reference to certain aspects thereof, other versions are possible. Embodiments disclosed herein are for illustration purposes only and various modifications of the embodiments and further embodiments thereof, in addition to those shown and described herein, will become apparent to those skilled in the art from the full contents of this specification.

1. A tablet composition comprising:
a spray dried dispersion (SDD) of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; and
a pH modifier.

2. The composition as claimed by claim 1 wherein the pH modifier is selected from the group consisting of calcium carbonate, magnesium carbonate, sodium bicarbonate, sodium carbonate, magnesium hydroxide, calcium hydroxide, magnesium oxide, and a mixture thereof.

3-4. (canceled)

5. The composition as claimed by claim 2 wherein the pH modifier is sodium carbonate.

6. (canceled)

7. The composition as claimed by any one of claims 1, 2, and 5, wherein the composition comprises:
a spray dried dispersion (SDD) of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier; and
a super disintegrant.

8. The composition as claimed by claim 7 wherein the super disintegrant is selected from the group consisting of croscarmellose sodium and crospovidone.

9. (canceled)

10. The composition as claimed by any one of claims 1, 2, 5, and 8, wherein the composition comprises:
3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
a pH modifier;
a super disintegrant; and
a lubricant.

11. (canceled)

12. The composition as claimed by claim 10 further comprising an immediate release coating.

13-14. (canceled)

15. The composition as claimed by any one of claims 1, 2, 5, 8, and 12, wherein the 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof, is 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of between about 0.7 to about 50 mg, on a free acid basis, per tablet composition.

16-21. (canceled)

22. A tablet composition wherein the composition comprises:
a spray dried dispersion (SDD) of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof;
in an amount of between about 0.7 mg to about 60 mg, on a free acid basis;
a pH modifier selected from the group consisting of calcium carbonate, sodium bicarbonate, sodium carbonate, sodium carbonate hydrate, magnesium hydroxide, and a mixture thereof; in an amount of about 2 mg to about 100 mg;
a super disintegrant selected from the group consisting of croscarmellose sodium and crospovidone; in an amount of about 2 mg to about 200 mg; and
a lubricant which is magnesium stearate; in an amount of about 0.1 mg to about 2.5 mg.

23. (canceled)

24. The tablet composition as claimed by claim 1 wherein the composition comprises:
the SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or a pharmaceutically acceptable salt thereof; in an amount of about 0.5 mg to about 75 mg, on a free acid basis;
and the balance of the SDD is composed of PVP-VA; and
a pH modifier selected from the group consisting of sodium bicarbonate and sodium carbonate; in an amount of between about 1 mg to about 150 mg.

25. The tablet composition as claimed by claim 24 wherein the composition comprises:
the SDD of about 30 wt % to about 35 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or the 0.5 Ca hydrate thereof; in an amount of between about 0.7 mg to about 60 mg, on a free acid basis; and the balance of the SDD is composed of PVP-VA; and
a pH modifier selected from the group consisting of sodium bicarbonate and sodium carbonate; in an amount of between about 5 mg to about 100 mg.

26. The tablet composition as claimed by claim 24 wherein the composition comprises:
the SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, or the 0.5 Ca hydrate thereof; in an amount of between about 1.7 mg to about 250 mg of the SDD; and the balance of the SDD is composed of PVP-VA; wherein the SDD is between about 2 wt % to about 25 wt % of the total tablet weight; and
the pH modifier which is sodium carbonate; in an amount of between about 6 mg to about 100 mg; wherein the pH modifier is between about 5 wt % to about 15 wt % of the total tablet weight.

27-28. (canceled)

29. The tablet composition as claimed by claim 26 wherein the composition comprises:
the SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; in an amount of between about 150 mg to about 250 mg of the SDD; and the balance of the SDD is composed of PVP-VA; wherein the SDD is between about 21 wt % to about 25 wt % of the total tablet weight; and
the pH modifier which is sodium carbonate; in an amount of between about 57.1 mg to about 80 mg; wherein the pH modifier is about 8 wt % of the total tablet weight.

30. The tablet composition as claimed by any one of claims 22, 24-26 and 29, wherein the SDD has a mean particle size of about 5 μm to about 113 μm in diameter; and
the pH modifier is sodium carbonate.

31. The tablet composition as claimed by claim 30, wherein the composition further comprises:
a super disintegrant selected from the group consisting of croscarmellose sodium and crospovidone.

32. The tablet composition as claimed by claim 31, wherein:
the super disintegrant is crospovidone in an amount of between about 8.5 mg to about 170 mg; wherein the super disintegrant is between about 10 wt % to about 17 wt % of the total tablet weight.

33. The tablet composition as claimed by claim 32, wherein the composition further comprises:
a filler which is MCC; in an amount of between about 67 mg to about 495 mg; wherein the filler is between about 49.5 wt % to about 79.5 wt % of the total tablet weight.

34. The tablet composition as claimed by claim 33, wherein the composition further comprises:
a lubricant which is magnesium stearate; in an amount of between about 0.4 mg to about 5 mg; wherein the lubricant is between about 0.1 wt % to about 1 wt % of the total tablet weight.

35. The tablet composition as claimed by claim 26, wherein the composition comprises:
the SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; and the balance of the SDD is composed of PVP-VA; wherein the SDD is about 19.5 wt % to about 25 wt % of the total tablet weight;
the pH modifier which is sodium carbonate; wherein the pH modifier is about 8 wt % of the total tablet weight;
a super disintegrant which is crospovidone; wherein the super disintegrant is about 17 wt % of the total tablet weight; and
a filler which is MCC; wherein the filler is about 49.5 wt % to about 55 wt % of the total tablet weight.

36-37. (canceled)

38. The tablet composition as claimed by claim 26, wherein the composition comprises:
the SDD of about 30 wt % of 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate; and the balance of the SDD is composed of PVP-VA; wherein the SDD is about 2.0 wt % to about 16.9 wt % of the total tablet weight;
the pH modifier which is sodium carbonate; wherein the pH modifier is about 8 wt % of the total tablet weight;
a super disintegrant which is crospovidone; wherein the super disintegrant is about 10 wt % of the total tablet weight; and
a filler which is MCC; wherein the filler is about 64.6 wt % to about 79.5 wt % of the total tablet weight.

39-44. (canceled)

45. The tablet composition as claimed by any one of claims 35 and 38, wherein the SDD has a mean particle size of about 5 μm to about 113 μm in diameter.

46. (canceled)

47. The tablet composition as claimed by any one of claims 22, 24-26, 29-35, 38 and 45, wherein the composition further comprises a lubricant which is magnesium stearate; wherein the lubricant is about 0.5 wt % of the total tablet weight.

48. The tablet composition as claimed by claim 47 wherein the composition further comprises an immediate release coating.

49-51. (canceled)
